(19) United States
(12) Patent Application Publication
(10) Pub. No.: US 2008/0033521 A1
(43) Pub. Date: Feb. 7, 2008

(54) Double-Wire Non-Trapping Angioplasty Catheter

(76) Inventors: Erik Jorgensen, Copenhagen (DK); Henning Kelbaek, Hellerup (DK); Steffen Helqvist, Bagsvaerd (DK)

Correspondence Address: KNOBBE MARTENS OLSON & BEAR LLP, 2040 MAIN STREET, FOURTEENTH FLOOR, IRVINE, CA, 92614, US

(21) Appl. No.: 10/586,011

(22) PCT Filed: Jan. 14, 2005
(86) PCT No.: PCT/DK05/00020

§ 371 Date: Oct. 18, 2007

(30) Foreign Application Priority Data
Jan. 16, 2004 (DK) PA 2004 00054

(51) Int. Cl. A61F 2/06

(52) U.S. Cl. 623/1.11

(57) ABSTRACT

The invention provides a catheter, which can be introduced into the coronary arteries over two or more wires. The catheter can be used for coronary, as well as for peripheral angioplasty, in particular near bifurcations. The catheter is of the rapid-exchange or over-the-wire type. The catheter allows procedures, in which two or more wires are introduced into the different branches of a bifurcation prior to positioning a stent proximal to the bifurcation. The wires are fully functional through the interior passage of the stent after its positioning. The procedure allows angioplasty of the distal vessels of the bifurcation without training or repositioning of the wires. The catheter thus avoids the problem of wire trapping between the stent and the artery wall. According to a method of the invention, a first stents is first positioned in the principal vessel and further conventional stents are positioned in the branches of the bifurcation with their proximal ends parts residing inside the first stent in the principal vessel.

### FIELD OF THE INVENTION

The invention relates to balloon and stent catheters and methods for coronary or peripheral angioplasty. More particularly, the invention relates to positioning of stents in and near artery bifurcations in patients with coronary artery disease. Though, also the invention is relevant for the insertion of stents in any bifurcating tubular vessel or organ, where stents are used or considered used such as the pulmonary veins, the bronchi, the gall duct etc.

### BACKGROUND OF THE iNVENTION

Coronary angioplasty is a medical procedure used to widen narrowed (stenosed) or open occluded coronary arteries, i.e. arteries supplying the heart muscle with oxygenated blood. Coronary angioplasty is used in patients with coronary artery disease (CAD). CAD is the most common type of heart disease and is the leading cause of death in the U.S. In both men and women. CAD occurs when the coronary arteries narrow or occlude due to the build-up of plaque in the wall of the arteries (atherosclerosis). The plaque consists of fat, fibrous tissue and may contain calcium deposits. Insufficient blood flow/oxygen supply to the heart muscle causes chest pain and/or dyspnoea. Acute thrombosis may occur in relation to plaque rupture resulting in abrupt closure of the artery and a subsequent myocardial infarction. For this situation acute coronary angioplasty has become an increasingly world wide used therapy.

Angioplasty (‘anglo’ is from the Greek word for vessel, and ‘plasty’ is from the Greek word for shaping), was first used in 1977. Since then, new devices and medications have improved the procedure and made the use of angioplasty appropriate in more cases of coronary artery disease. The improvements include the implantation of stents. A stent is a tiny mesh-like tubular metal scaffolding. It is crimped onto a non inflated balloon and under balloon dilatation deployed at the diseased site in the coronary artery, either after pre-balloon dilatation or directly without predilatation. After stent deployment the balloon is deflated and removed, whereas the stent will remain in close apposition with the inner arterial wall. Coronary angioplasty is performed during local anaesthesia: a guiding catheter is passed through the skin into the femoral or radial artery. The guiding catheter passes against the blood stream through the aorta reaching the origin of the coronary artery just above the aortic valve. Under fluoroscopic guidance with the use of X-ray contrast media a steerable guidewire is introduced through the guiding catheter out into the coronary artery and through the stenosed/occluded segment. A balloon-catheter with or without a stent is threaded and advanced over the guidewire to the stenosed/occluded site. The balloon is then expanded by fluid to displace the plaque against the arterial wall, thereby widening the artery and increasing blood flow. The use of stents serves several purposes: 1. stenting reduces acute complications during angioplasty as dissection and closure of the artery due to injury induced by balloon dilatation, 2. stenting results in a lesser degree of residual stenosis compared to balloon dilatation alone, and 3. stenting results in a lower frequency of late restenosis. On the other hand, stenting induces an inflammatory process in the arterial wall resulting in a sometimes exaggerated proliferation of a scar like tissue creating an “in stent restenosis”. To overcome the latter problem stents coated with anti proliferative medical agents have been available since 2002.

A highly important feature of the process of angioplasty is the placement and presence of the thin wire (typically 0.014″ in diameter) over which the balloon catheter is threaded. The difficulty of advancing a wire through a diseased arterial segment varies according to the characteristics of the artery and the stenosis. Balloon dilatation will cause various degrees of mechanical destruction of the arterial wall. The wall destructive action of the balloon dilatation will often make a subsequent advancement of a wire through the segment very difficult or even impossible. Therefore, the wire should never be removed until the whole process has been finalised. The worst scenario of balloon mediated wall destruction is dissection of the arterial wall resulting in closure of the artery with a total cut off of blood flow. This situation is often easy to handle—but only with the wire in place—by advancing a balloon catheter with a stent to the dissection/occlusion site and deploy the stent, thereby sealing the dissection and restoring blood flow. The crucial point in this process is the stable presence of the wire through the segment of interest, making further angioplasty with stenting possible. When the stenosis is located to a segment where a sidebranch leaves a main branch (bifurcation) it is obligatory to advance two wires in order to treat both branches. If stenting of the main branch proximally or partly proximally to the origin of the side branch is subsequently performed with the two wires present, the side branch wire will be trapped or jailed between the stent and the arterial wall. As explained, if one wire is withdrawn before stenting—to avoid jailing—it might be impossible to advance this wire after stenting, thus treatment of the bifurcation will not be possible. Present catheters are either ‘Rapid Exchange’ (RX)=Monorail type catheters (which allow removal and exchange of the catheter using short wires) or ‘Over-the-wire’ (OTW) catheters (which allow exchange of either the wire or the catheter). There exist a number of techniques and devices for stenting branches in bifurcations, which can generally be assigned to either Y-stenting or Y-stents or to T stenting.

### Y-stenting

Y-stenting is a method of simultaneously implanting individual stents in each branched vessel of a bifurcation. The stents may reside completely within the branch or may have their proximal ends in the principal vessel, where they overlap side-by-side. Such methods as well as stents and catheters applied in such methods are described in e.g. U.S. Pat. No. 5,632,763, U.S. Pat. No. 6,599,315, US 2002/0143383, and EP 0 347 023.

If the principal vessel proximal to the bifurcation also suffers from atherosclerosis, this section might be considered stented as well. This may be done by using long stents for the branch vessels and letting a proximal end of these stents extend through the blocked section of the principal vessel. This is illustrated in FIG. 1A. It is a disadvantage of this technique that the Y-stenting results in a long section of two side-by-side stents in the principal vessel. Firstly, this creates a long segment of a double stent “barrel” interfering with blood flow and thereby increasing the risk of stent thrombosis. Secondly, at implantation, one stent may exert a collapsing pressure on or cut off the other if they do not extend side-by-side all the way. This is illustrated in FIG. 1B.

### Y-stents

There have been several attempts to produce a Y-shaped stent which could stent both branches and principal vessel of a bifurcation in one go. Such ‘Y-stents’ are typically shaped like a pair of trousers with legs for each branch and a larger diameter waist for the principal vessel. Disclosures of Y-stents and methods for implanting Y-stents may be found in e.g. U.S. Pat. No. 4,994,071, US 2002/0111675, US 2001/0029396, US 2002/0049412, and US 2002/0058905.

Y-stents have not provided any breakthrough in coronary angioplasty. In fact, the testing have shown so poor results that no Y-stents are presently available on the market. It is a major disadvantage that Y-stents lack versatility in relation to the shape of the bifurcation, they either fit or they do not. The cardiologist will need an extremely large selection of Y-stents to choose from in order to select the one having the appropriate geometry for the given situation. Often, one branch may be more blocked with plaque than the other, in which cases the two legs of the Y-stent should have different diameters and lengths.

It is another disadvantage that the actual diameters and angles of the branched vessels are difficult to estimate, when thick layers of plaque cover the wall of the main vessel. Hence, whether the selected Y-stent was correct will not be apparent until after the final positioning of the Y-stent, which might be too late.

### T-stenting

With the T-stenting technique, a stent is initially implanted in the main branch covering the origin of the side branch. Thereafter, a guidewire is brought between stent struts into the side branch. After balloon dilatation between struts another stent is deployed in the side branch from the its origin and distally. Alternatively, the side branch stent may be deployed before main branch stenting. Both techniques optimally involve a finalising simultaneous balloon dilatation of both main and side branch (“kissing balloons”) in order to achieve a sufficient apposition of stents to the arterial wall.

T-stenting has several drawbacks. Most important is the risk of sidebranch occlusion and subsequent difficulties in wire positioning through the stent struts into the occluded vessel. Secondly, the deformation of the main branch stent when the sidebranch is accessed and balloon dilated will influence blood flow and increase the risk of thrombosis. Thirdly the accurate positioning and expansion of the sidebranch stent is difficult and will always lead to some stent protrusion into the main lumen, alternatively to an area of the vessel wall without stent coverage. Kissing balloons dilatation is not always technically possible.

### Dilation Catheters

WO 03/074118, FR 2740346, and WO 94/16761 disclose dilation catheters used to dilate blocked arteries without stent implantation. In WO 03/074118, FR 2740346, the catheter is split to provide different balloons, or different parts of the same balloon, to enter each branch vessel of the bifurcation. WO 03/074118 does mention that the catheter may be used to introduce stents such as Y-stents. It is a disadvantage that these catheters enter the branches at all. As the branches may have different sizes, a very large number of catheters with pairs of different sized balloons must be available to the cardiologist.

WO 94/16761 describes a dilatation catheter for inserting successive dilatation balloons. The catheter is an old type catheter that cannot be withdrawn or exchanged without also removing the guidewire, i.e. not a RX or an OTW type catheter. It is a major disadvantage that the catheter cannot be removed independently of the guidewire. In cases of balloon induced dissection of the vessel wall, reinsertion of a guidewire might turn out to be extremely difficult/impossible. For this reason, the guidewire is best kept in the dilated vessel until a stable and open lumen is observed.

The problem of stenting in the bifurcation and proximal and distal to the bifurcation still remains to be solved, as there is presently no method or system available on the market which provides a safe and efficient solution. There has, as the prior art mentioned in the above shows, been a number of attempts, but none which functions satisfactorily in terms of improving patients' symptoms or length of life. There is a need for improved methods and tools for performing coronary angioplasty in vessel bifurcations and proximal to vessel bifurcations. The trend in the prior art is to make more complex Y-stents and corresponding Y-shaped balloon catheters for implantation of these at bifurcations in easy one-step stenting procedures.

### SUMMARY OF THE iNVENTION

In practising as cardiologists, having performed thousands of coronary angioplasty procedures, the inventors of the present invention have a large experience and a deep practical understanding of the problems involved in stenting bifurcations and vessels proximal to bifurcations.

The inventors have realised that due to the multiplicity of artheroscierotic narrowing in relation to bifurcations of the coronary arteries, a simpler, more versatile solution is needed which provides a large number of options for adapting existing coronary angioplasty devices to the situation at hand.

It is an object of the invention to provide a catheter that allows implantation of a stent over two or more wires. Such a catheter can stent the main vessel proximal to the bifurcation while having guidewires through the stent into all branched vessels, without leaving a wire trapped between the stent and the vessel wall. This allows subsequent stenting of the vessel branches without removing and replacing one of the guidewires.

It is another object of the invention to provide a novel stenting procedure where, after guidewires have been advanced into both branched vessels of the bifurcation, a principal stent is implanted in the principal vessel over both guidewires. Thereupon further stents are positioned in the branched vessels, with their proximal parts residing inside the first stent in the principal vessel, thereby creating complete stent coverage of the vessel wall of the bifurcation.

The order of succession in which the stents are implanted and their spatial overlap according to the present invention is essential. Stenting the principal vessel first, followed by “kissing stenting” of the main and side branches with a very short double stent barrel protruding into the distal part of the single proximal stent allows a complete restoration of the artery wall of the whole bifurcation segment.

In contrast, deployment of the kissing stents before deployment of the single proximal stent will either result in a destruction of the double stent barrel structure caused by the dilated last, proximal stent balloon (since it is mounted on only one wire), or a non stented gap will be left between the distal part of the proximal stent and the double stent barrel. The latter is illustrated in FIG. 2A. It is considered a major disadvantage that this interspace cannot be stented, due to the fact that the unstented artery wall at and proximal to the bifurcation might restenose and influence blood flow, because of recoil or obstructing dissections.

If the stents are implanted in the order prescribed by the invention using conventional balloon catheters and techniques, the following problem arises: Due to the possible disrupture of the arterial wall at the proximal and distal stent-end, it is desirable to place all guidewires prior to implanting the first stent. Therefore, guidewires in both branches should be put in place initially. The traditional way of stenting the principal vessel using a standard catheter, advanced over only one of the guidewires, results in jamming of the other wire between the stent and the artery wall. This is illustrated in FIG. 2B. Obviously, the jammed guidewire cannot be used to advance a catheter into a branch and a new guidewire must be introduced between the stent struts into the side branch. As the artery wall now might be disrupted, finding an entry into the side branch might turn out to be difficult and time consuming, or might even be impossible. Therefore, this method has an increased risk of introducing complications due to side branch closure. If initially only one guidewire in the main vessel is used, a second guidewire has to be introduced into the side branch after stenting of the principal vessel. In this case there is an equally increased risk of untreatable side branch closure.

It is a fact of the state of the art that the principal vessel cannot be stented prior to the branched vessels to create an overlap and avoid unstented interspace between stents, without also getting wire-trapping and untreatable side branch closure.

The present invention overcomes those difficulties by providing the following advantages.

It is an advantage of the invention that jamming, squeezing or trapping of a guidewire between the outside of the stent and the vessel wall is avoided.

It is another advantage that stents in branched vessels only have a short overlap with the stent in the principal vessel, since the presence of a long double stent barrel is a problem for blood flow. Without the proximal stent, a long double stent barrel would have been necessary in case of a long diseased proximal segment.

It is another advantage of the invention that it makes possible angioplasty of the principal vessel proximal to the bifurcation with wires in place that secure easy and uninterrupted access to both distal branches in cases of vessel wall dissections with subsequent occlusions.

It is another advantage of the invention that it can be easily adapted to all vessel sizes and angles of branches. Also, the invention provides freedom of choice in the length and diameter of the stents in principal and branched vessel. Furthermore, the invention applies the use of standard stents for the distal vessel branches, and thereby takes advantage of the present broad available selection of stents with different lengths, diameters, material, bending flexibility, strength, etc.

It is yet another advantage of the invention that the presence of the proximal stent prevents a dissection caused by the kissing stenting.

It is a further advantage of the invention that it is possible to perform a subsequent angioplasty at the proximal stent edge (due to either an acute dissection at the proximal stent edge or a later development of a stent edge stenosis), without destroying the double stent barrel structure close to the bifurcation.

In a first aspect, the invention provides a catheter system for positioning of a stent in an angioplasty procedure. The catheter provides passage for two or more wires through the inside and along the length of the stent to be positioned. As the object of the invention may be implemented in several different configurations and as there exist several different kinds of catheters for stent positioning, the first aspect is directed to different implementations.

Within a first implementation of the first aspect, the invention provides a balloon catheter for positioning of a stent in coronary or peripheral angioplasty procedures. The catheter comprises a hollow conduit with an open proximal end and a closed distal end and having an expandable section with an outer surface part adapted to hold a stent and having no bifurcations or side openings, and one or more guidewire lumens or grooves to provide passage for two or more guidewires through a stent to be held by said outer surface part of the expandable section. The catheter may be an over-the-wire or a rapid exchange type catheter or both as the catheter may comprise two or more different guidewire lumens.

In a preferred embodiment, the one or more guidewire lumens provide passage for two or more guidewires inside said outer surface part of the hollow conduit, the guidewire lumen(s) extending from opening(s) through the closed end of the hollow conduit distal to said outer surface part (inlet(s)), and to one or more openings proximal to said outer surface part (outlet(s)). As is obvious to the skilled person, the guidewire lumens are not in fluid connection with the hollow conduit. In this embodiment, the guidewire lumen(s) provide(s) passage for two or more wires inside an outer perimeter of the hollow conduit, at least along the length of the part of the balloon which is to hold the stent.

A second implementation of the first aspect provides a balloon catheter for positioning of a stent in coronary or peripheral angioplasty, the catheter comprising a hollow conduit with an open proximal end and a closed distal end and having an expandable section for holding and expanding a stent, the balloon catheter being characterised in that
it further comprises one or more guidewire lumens or grooves extending along at least part of the expandable section and providing passage for at least two guidewires inside the expandable section so that, after expansion of a stent by the expandable section, the at least two guidewires run through the stent from end to end, and in that
the expandable section has an outer perimeter with no bifurcations or side openings.

A third implementation of the first aspect provides a similar balloon catheter, being characterised in that
it further comprises one or more guidewire lumens or grooves extending along at least part of the expandable section and providing passage for at least two guidewires inside the expandable section so that, after expansion of a stent by the expandable section, the at least two guidewires pass through the stent from end to end, and in that
it is adapted to position the stent in a principal vessel proximal to the bifurcation without entering either branch distal to the bifurcation with the expandable section.

It is thereby expressed that a main objective of the catheter according to the first aspect is to allow for implanting of a stent while providing two guidewires through the stent, while allowing for the use of standard stents and a limited number of catheters. Thus, the cardiologist need not consider the geometry and condition of the bifurcation when choosing the catheter, as the balloon does not enter the branches. Preferably, the choice of catheter depends solely on the conditions proximally to the bifurcation, such as the diameter of the principal vessel and the extent of the stenosis proximally to the bifurcation. This provides an enormous advantage over bifurcated and otherwise customised catheters of the prior art, in that the cardiologist need not stock a huge number of custom catheters depending on the specific geometry and condition of the vessels.

Within a second class of catheters, mounted with self-expandable stents, the first aspect of the invention provides a catheter for positioning of a self-expanding stent in coronary or peripheral angioplasty, the catheter being an over-the-wire and/or a rapid exchange type catheter comprising a self-expanding stent mounted on a distal end section of the catheter, a sheath keeping the self-expanding stent in a compressed state, and one or more guidewire lumens providing passage for two or more wires through the inside of the self-expanding stent.

Preferably, the distal end section of the catheter and/or the stents mounted or to be mounted thereon, has no bifurcations. Also, the one or more guidewire lumens preferably lie within an outer perimeter of the catheter.

The term inside or within an outer perimeter of the catheter means that a lumen is not a separate lumen attached to the outside of the catheter body, projecting outside the outer perimeter of the catheter. Such an attached lumen, although it may be applicable, is not preferred as it may make it difficult to securely attach the contracted stent to the catheter (deflated balloon) because of its irregular shape. Also, it may deform the expanded stent in case of a balloon expansion. According to the invention, a lumen may reside inside the catheter or may be integrated in the outer wall of the catheter. A lumen may also be embedded in a trench or groove in the wall of catheter, preferably so that it does not project outside an interpolation of the outer perimeter of the catheter, i.e. extending out from the groove in the outer perimeter. In yet another alternative, the lumen may be implemented by a groove in the catheter or balloon, over which groove the stent is mounted. It is to be understood, that when the term opening is used in relation to the end part of a guidewire lumen, it shall also apply to the end part of a groove performing the function of a guidewire lumen although it does not present an opening as such. When a stent is mounted over the groove, the rim of the stent together with the edges of the groove will provide an opening as such. In the remaining text, the term lumen is used as also covering a groove unless the opposite is implied.

The term that a section of the catheter has no bifurcations (branches or splits) or side openings (besides the openings at the ends), means that the catheter is as simple as possible in order to also simplify its use and the risk of complications. The various catheter sections preferably have at least substantially tubular outer perimeters.

The term ‘principal vessels’ refer to the vessel or artery before (or after, depending of the direction) the bifurcation. The term branched vessels refer to the vessels emerging from (or converging into, depending on the direction) the principal vessel. Hence, in this terminology, a small side branch to a large vessel means that the section of the large vessel above the side branch is the principal vessel and that the side branch and section of the large vessel below the side branch are branched vessels.

Cardiac catherterisation laboratories typically have catheters with about ten different stent lengths in ten different diameters, amounting to 100 different stent geometries, to match the majority of vessel geometries. It would be extremely expensive if a clinic should keep Y-stents with corresponding Y-shaped catheters in such a diversity of geometries and sizes. The method according to the invention combines three stents to rebuild the wall of the bifurcation. The selection of stents already present in catheterisatlon laboratories offers thousands of possible combinations to fit any bifurcation. The catheters according to the invention therefore provide an extremely simple solution which is economically much more attractive than the solutions provided by the prior art. According to the method of the invention, each stent is a standard stent positioned similarly to the way stents are positioned today using a catheter that is operated identically to existing catheters. Thus the method provides an extremely easy and safe solution which cardiologists can adopt immediately without additional training.

The catheter according to the invention might also be used as a normal catheter with only one wire. This might be of particular interest in large vessels, in which the profile of the catheter is of less importance. Replacing the normal catheters with the invention for large arteries will limit the total number of catheters with mounted stents needed in a laboratory performing bifurcation angioplasty.

The one or more guidewire lumens of the catheters according to the first aspect must provide passage for two or more guidewires. Most often, only two guidewires are needed and when the present description makes reference to two guidewires, two or more may apply unless the opposite is implied. Present catheters cannot be threaded with more than one guidewire. The reason being that catheters are constructed to precisely fit the standardised guidewire diameters, 0,014″ or 0,018″ in order to reduce the profile of the catheter tip, and thereby facilitating the introduction of the catheter into narrow arteries. It is simply not possible to fit more than one wire along the length of the lumen of a catheter.

The catheters according to the invention may have only one lumen large enough to provide passage for two guidewires or more lumens each providing passage for one guidewire. Several single-wire lumens are preferable as this considerably reduces the risk of guidewire entanglement inside and outside the lumen.

The one or more lumens may be formed to provide a combinatory OTW and RX type catheter. In a preferred embodiment, a first guidewire lumen extends inside an outer perimeter of and along the length of the catheter to form an OTW type lumen, whereas a second guidewire lumen is shorter than the catheter and has an open distal end at the distal end of the catheter and an open proximal end part positioned proximal to, but nearby, the section holding the stent to form a RX type lumen. In other embodiments, the lumens of a catheter may be solely OTW or RX type lumens.

The various implementations of the first aspect specifies the overall layout of the catheter. As stenosis or occlusion are often found at the bifurcation, it is an object of the invention to allow positioning of the first stent very close to the bifurcation since this best imitates the layout of the bifurcation. The design of the distal tip of the catheter is important as this may determine how close to the bifurcation the stent can be positioned. It is important to bear in mind that guidewires coming from the catheter (distally to the stent) must be able to enter both branches so that it may not be possible to simply introduce the catheter tip into one branch to get the stent close to the bifurcation.

A short catheter tip allows the stent in the principal vessel to be mounted close to the bifurcation while keeping guidewires in both branches and not entering one of the branches. This typically means that the tip will have a high profile, giving the catheter a much more blunt distal end. Such blunt distal end is not considered a disadvantage as this catheter is preferably used in the more spacious principal vessels of the coronary arteries. This is contrary to the trends in designing balloon catheters, which go toward making longer, tapered tips with as low profile as possible to ensure easy access to all lesions. However, if the bifurcation branches should be stented after having positioned the stent proximal to the bifurcation, such low profile catheter may be preferred as the branch may be ruptured.

In an alternative to a short tip, one of the wires may simply leave the catheter through an opening between the distal tip of the catheter and the distal end of the surface part of the balloon adapted to hold the stent. Thereby, the tip of the catheter may enter one branch up to the exiting point of the other guidewire.

Thus, in a preferred embodiment, the expandable section of the balloon catheter preferably comprises tapered end sections and a cylindrical central section for holding a stent, where a distance from the distal end of the cylindrical central section to an inlet of a first guidewire lumen or groove is less than 8 mm, such as less than 6 mm, such as less than 5 mm, such as less than 4 mm, such as less than 3 mm, or less than 2 mm. This allows for the stent to be positioned 8, 6, 5, 4, 3, or 2 mm from the bifurcation, respectively. In this case, the length of the remaining tip of the catheter is not important and it may enter a branch.

In another embodiment, the tip may be longer and provide no inlets or openings other than at the extreme distal end. This may be realised by a split tip, essentially consisting of two or more tubes emerging from close to the distal end of the balloon and providing individual guidewire lumens. Therefore, in a further preferred embodiment of the first aspect, the one or more guidewire lumens of the balloon catheter extends beyond an extreme distal end of the balloon and is divided into two or more individual guidewire lumens at a position of exit from the extreme distal end of the balloon.

In all of the above embodiments for catheter tip designs, it is the point of split-up between the guidewires, or their lumen(s), which is important. This point of split-up is preferably situated as close to the distal end of the cylindrical central section, or the stent, when mounted, as possible. This will be clearly illustrated in relation to FIGS. 8A-C later on.

Often, balloon catheters are sold with a stent mounted on the balloon. Therefore, in a preferred embodiment, the invention provides an assembled stent delivery system. The assembled stent delivery system preferably comprises a balloon catheter as described in relation to the first aspect with a stent held by the expandable section so that the one or more guidewire lumens or grooves provides inlets and outlets for two or more guidewires distally and proximally to the stent. Upon threading the catheter, the guidewires will enter the inlet(s) of the lumen(s) distally to the stent and exit the outlet(s) of the lumen(s) proximally to the stent as the catheter is introduced. After expansion of the stent, and withdrawal of the catheter, both guidewires will pass through the stent while remaining in the bifurcation branches.

In a second aspect, the present invention provides an assembled stent delivery system comprising two or more balloon catheters extending in parallel and holding a shared stent, the system thereby providing passage for two or more guidewires through the shared stent. Preferably, the shared stent is held by and circumvents expandable sections of at least two balloon catheters, or an expandable section of a first balloon catheter and a non-expandable section of a second balloon catheter. These arrangements are illustrated in FIGS. 7A-B. The catheters may be over-the-wire and/or rapid exchange type catheters.

The stents applied in the various aspects of the present invention may be coated with anti-proliferative medical agents, or be drug eluting stents. Alternatively, or supplementary, the stents may be bio-degradable or bio-absorbable stents.

In a third aspect, the present invention provides a method for positioning a stent in a principal vessel proximal (or distal) to a bifurcation while having guidewires in branches of the bifurcation. The method comprises the steps of:
inserting a distal end of a first guidewire through the principal vessel and into a first branch of the bifurcation,
inserting a distal end of a second guidewire through the principal vessel and into a second branch of the bifurcation,
providing a first catheter for positioning of a first expandable stent mounted on a distal end section of the catheter, the first catheter comprising one or more guidewire lumens providing passage for two or more wires through the stent from end to end,
threading the one or more guidewire lumens with proximal ends of the first and the second wire,
advancing the first catheter simultaneously over the first and the second wire until the first stent reaches the principal vessel proximal to the bifurcation,
expanding the first stent.

The term ‘threading a guidewire lumen with a guidewire’ means inserting the guidewire into an open end of the lumen and advancing it inside and along the length of the lumen until at least part of the guidewire projects out from an opposite open end of the lumen.

The method according to the third aspect preferably applies one of the catheters of the first aspect. After withdrawing the first catheter simultaneously over the first and the second wire, the stent in the principal vessel is left with both guidewires passing through it and into the bifurcation branches. At this point, the cardiologist can decide whether to implant stents in the branches, implant further stents in the principal vessel proximally to the first stent, or whether the situation is stable. Any further stenting can be carried out without complications as the required guidewires are already fully advanced in the branches. The choice depends upon the degree of stenosis or occlusion and the result of proximal stenting. The catheters and method according to the invention may thus be used in a single stent procedure.

The application of the catheter according to the first aspect, the systems according to the second aspect and the method according to the third aspect provide the cardiologist with extra security and added possibilities. Sometimes, closure of one of the distal bifurcation branches is experienced after implanting of the first stent or additional proximal stents in the principal vessel. In this case, the guidewires already advanced allow for a fast and uncomplicated stenting of the branch in question. Therefore, upon additional proximal stenting in the principal vessel, both guidewires should remain and the dual-lumen catheter according to the invention should be applied. Thereby, the option for further distal stenting is preserved at any later time.

The method may further comprise positioning a stent in one, first branch of the bifurcation by further comprising the steps of:
withdrawing the first catheter simultaneously over the first and the second wire,
threading and advancing a second catheter mounted with a second expandable stent over the first guidewire and at least partially into the first branch of the bifurcation,
expanding the second stent of the second catheter.

The method may then further comprise successive positioning of a stent in a second branch of the bifurcation by further comprising the steps of:
threading and advancing a third catheter mounted with a third expandable stent over the second guidewire and at least partially into the second branch of the bifurcation, and
expanding the third stent of the third catheter.

The second and the third stent may preferably be expanded at least substantially simultaneously.

Preferably, the stents at the bifurcation are positioned so as to overlap side-by-side inside the stent in the principal vessel. For this purpose, the steps of advancing the second or third catheter may comprise:
advancing the second/third catheter so that a distal end of the second/third stent is positioned in the first/second branch of the bifurcation and a proximal end is positioned inside the first stent.

According to a fourth aspect, the present invention provides a method for fabricating a catheter according to the first aspect. Catheters according to the first aspect may be fabricated similar to the fabrication of present catheters by increasing the diameter of the catheter body and arrange an extra guidewire lumen.

According to a fifth aspect, the present invention provides the use of a catheter according to the first aspect for performing angioplasty.

According to a sixth aspect, the present invention provides a catheter according to the first aspect for use in coronary angioplasty on humans.

These and other aspects of the invention will be apparent from and elucidated with reference to the embodiment(s) described hereinafter.

### BRIEF DESCRIPTION OF THE DRAWINGS

FIG. 1A illustrates a Y-stented bifurcation. FIG. 1B illustrates a Y-stented bifurcation where one stent closes off the other.

FIG. 2A illustrates the interspace resulting when stenting the principal vessel proximal to a Y-stented bifurcation with prior art techniques and equipment, i.e. stenting of side branches before proximal stenting. FIG. 2B illustrates wire-trapping when stenting the principal vessel while having guidewires in all branches.

FIG. 3 shows an RX dual-wire lumen catheter according to the invention.

FIG. 4A shows a side view of a combinatory RX and OTW dual-wire lumen catheter according to the invention, FIG. 4B shows a cross-sectional view of the combinatory RX and OTW dual-wire lumen catheter.

FIG. 5A shows a dual-wire catheter (RX) according to the invention with a self-expanding stent in a compressed state, FIG. 5B shows the self-expanding stent during expansion.

FIGS. 6A-D illustrate the stenting method according to the invention.

FIG. 7A illustrates two dual-wire lumen catheters (OTW) positioned in substantially parallel relationship and having an expandable stent positioned on the expandable section of a first catheter which stent also covers part of the lumen of a second catheter.

FIG. 7B illustrates two dual-wire lumen catheters (OTW) positioned in substantially parallel relationship and having an expandable stent positioned around the side-by-side positioned expandable sections of both catheters.

FIGS. 8A-C illustrate different configurations of dual-wire lumens with inserted guidewires at a short end tip of a balloon catheter.

FIG. 9 illustrates a catheter with a split tip at the distal end of the balloon.

### DETAILED DESCRIPTION OF THE DRAWINGS

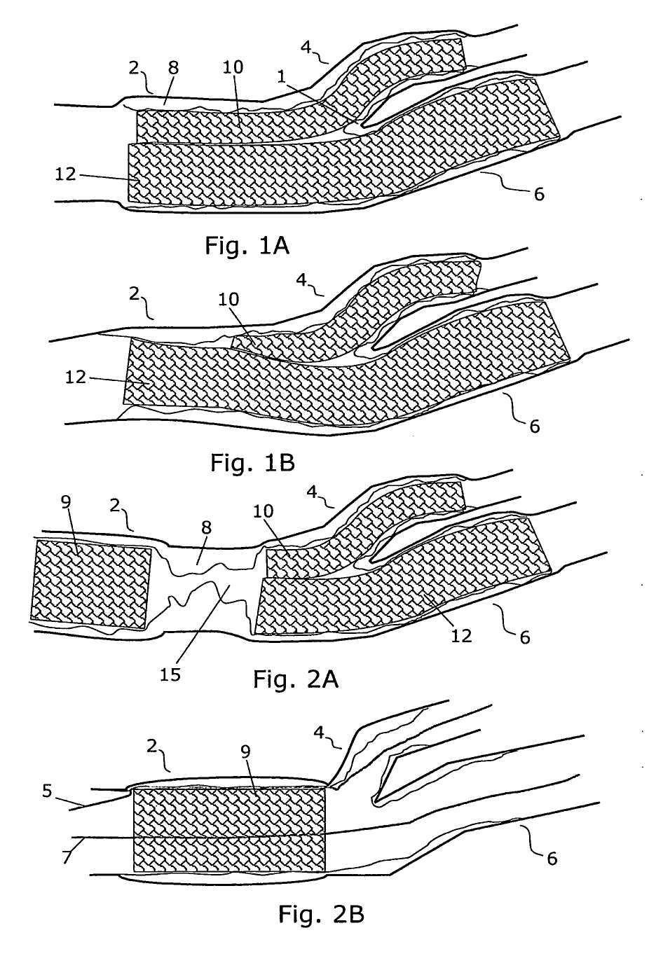

FIG. 1A illustrates a Y-stenting according to the prior art. Stents 10 and 12 are positioned and expanded by individual catheters so that their proximal end parts reside side-by-side in the principal vessel 2 and each distal end part reside in a branched vessel 4 and 6, respectively. If the principal vessel has plaque 8 far above the bifurcation 3, stents 10 and 12 will overlap over a long distance, which is not desirable. FIG. 1B illustrates the situation where the Y-stenting has resulted in the collapse of the stent 10.

FIG. 2A illustrates the interspace 15 arising when the branched vessels 4 and 6 are stented before stenting of the principal vessel 2. A heavy build-up of plaque 8 squeezed together by the stents 9, 10 and 12 threatens to clog the vessel.

FIG. 2B illustrates the situation where the principal vessel 2 is stented before the branched vessels 4 and 6, but after laying guidewires 5 and 7 into the branched vessels 4 and 6. Using a standard catheter to insert stent 9, advanced over either one of wires 5 or 7, results in trapping of the other wire (here 5) between the stent 9 and the vessel wall.

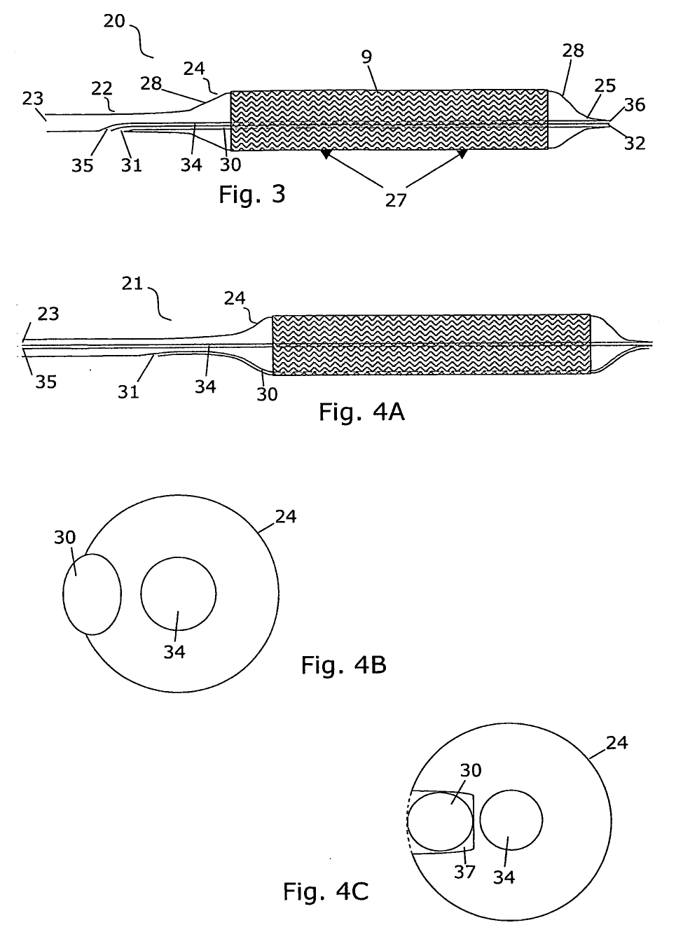

FIG. 3 shows a balloon catheter 20 according to a preferred embodiment of the Invention. The catheter has a hollow conduit 22 with an expandable section 24, the balloon. A proximal end part 23 of the conduit is open so that a fluid pressure can be applied to expand the balloon, correspondingly, a distal end part of the conduit, 25, is closed. An expandable stent 9 is mounted of the balloon, which is shown in its deflated state. The catheter has two guidewire lumens 30 and 34 providing passage for two or more guidewires through the balloon. The guidewire lumens 30 and 34 has open ends 31, 32 and 35, 36 proximal and distal to the balloon as shown.

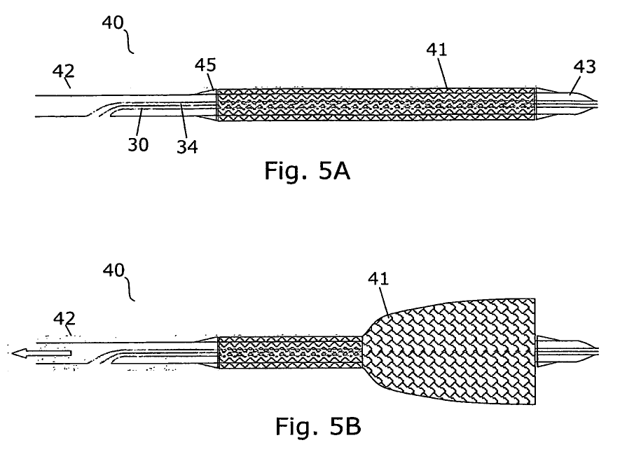

In the catheter shown in FIG. 3, both guidewire lumens 30 and 34 are of the RX (Rapid Exchange) type in that the proximal ends 31 and 35 resides just above the balloon 24. As shown in FIG. 4A, one catheter can be RX while another is OTW (Over the Wire) type where the proximal end 35 is positioned at the end part 23 of the conduit. The balloon has a cylindrical central section 27 to hold the expandable stent 9 and tapered end sections 28 on either side of the central section.

In another preferred embodiment shown in FIG. 4A, the lumen 34 extends inside the balloon 24 of catheter 21, whereas lumen 30 extends along the outer perimeter of the balloon 24. The lumen 30 can be integrated in the outer perimeter or wall of the balloon 24 as shown in FIG. 4B. Alternatively, the lumen 30 can be countersunk into a trench 37 formed in the outer perimeter of the balloon. In FIG. 4C, the lumen 30 in trench 37 is positioned within an interpolation of the outer perimeter over the trench (dashed line).

FIGS. 5A and B shows another kind of catheter 40 according to the invention. Here, catheter 40 is mounted with a self-expanding stent 41. The stent 41 is held in a compressed state by a withdrawable sheath 42 while in position by stops 45. When using a self-expanding stent 41, the catheter body 43 need not be a hollow conduit nor having an expandable section. Typically the catheter body 43 is a flexible plastic tubing. Withdrawing the sheath as illustrated by the arrow in FIG. 5B causes the stent 41 to expand. According to the invention, the catheter has lumens 30 and 34 providing passage inside the self-expandable stent 41. Similar to the embodiment described in relation to FIGS. 4A-C, the lumens may be RX or OTW and may extend inside the catheter body, be integrated in the wall or be countersunk in a trench.

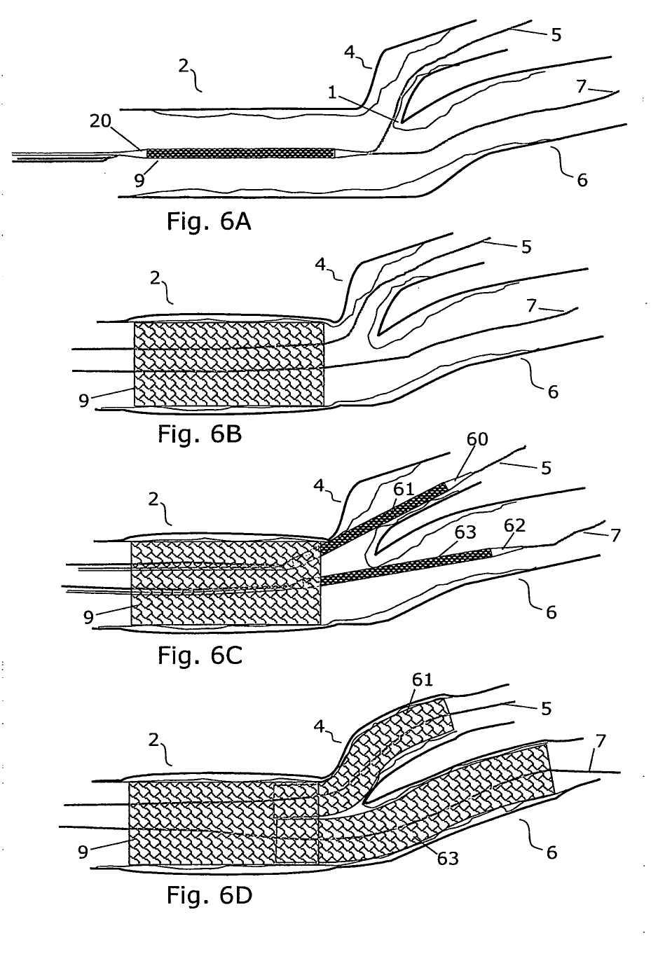

The method for stenting at and proximal to a bifurcation 1 according to the present invention is described in the following sections and is illustrated in FIGS. 6A-D. The method can be applied in different stenting procedures in arteries and tubular bifurcating structures such as the gall ducts, large glands a.o. but is described in relation to a coronary angioplasty using the balloon catheter 20 described in relation to FIG. 3.

First, guidewires 5 and 7 are laid in branched arteries 4 and 6, respectively. It is important that the guidewires are inserted prior to any stenting and that they stay in position during the entire procedure. Lumens 30 and 34 of the catheter 20 are threaded with wires 5 and 7, and the catheter is advanced over the wires to the principal artery 2, FIG. 6A. The catheter 20 is advanced until immediately proximal to the bifurcation. When the catheter is in position, the balloon is inflated to position stent 9 in the principal artery with wires 5 and 7 being inside the stent, FIG. 6B. Catheter 20 can now be withdrawn over wires 5 and 7, leaving both in their respective branched artery. This completes the stenting of the principal artery. It is obvious that the stent 9 can be chosen to have any appropriate length and diameter depending on the plaque build-up in the artery and the artery geometry. Also the construction of the stent might vary depending on the purpose of stenting. In general the stenting of a dissection flap does not need a high radial stent strength, whereas a calcified vessel might need a stent with high radial strength. In cases of increased restenosis risk a drug-eluting stent might be chosen. In unusual cases the stenting might aim to cover a perforation or vessel rupture or an aneurysm, in which cases a membrane covered stent could be chosen.

To stent the branched arteries, catheters 60 and 62 with stents 61 and 63 are threaded with and advanced over wires 5 and 7, respectively. Catheters 60 and 62 may be a catheter according to the present invention or a prior art catheter. Catheters 60 and 62 are advanced until distal ends of stents 61 and 63 reside inside a branch and proximal ends of stents 61 and 63 reside inside stent 9 in the principal artery, FIG. 6C. The distal stents should be positioned with a sufficient overlap to secure that no segment of the vessel remains uncovered of a stent. In general the operator should aim for one or a few mm of overlap to secure vessel wall coverage in the bifurcation. Proximally and distally the main vessel stent and side branch stents should in general cover all of the diseased segments according to the discretion of the operator.

In most cases the operator will optimise the flow conditions by aiming for an equal total artery lumen (cross-sectional area) before and after the bifurcation and the expected size of stent 9 is

D₉=√ D̅₆̅₁̅²̅+̅D̅₆̅₃̅²̅,

where Dₓ is the diameter of stent x in its expanded state, if there are no other large branches involved in the bifurcation. Alternatively, the stents 61 and 63 can be chosen so that they fit inside stent 9 when expanded, hence

D₉≧D₆₁+D₆₃.

In general, the calculation of the stent size by the operator should always take the present state of the pathology of the artery into consideration.

When catheters 60 and 62 are in position, the balloons are inflated to position stents 61 and 63. Catheters 60 and 62 can now be withdrawn over wires 5 and 7, leaving the wires in position, FIG. 6D. As can be seen, stents 61 and 63 overlap side-by-side inside stent 9. An overlap as described secures that no parts of the vessel wall protrude into the lumen. This is important as wall protrusion into the lumen (as illustrated in FIG. 2A) might increase the risk of thrombosis or so called sub acute stent thrombosis, a much feared complication of stenting. Also, stent overlap reduces the risk of vessel wall recoil and subsequent restenosis. After confirming a stable blood flow through the bifurcation, guidewires 5 and 7 are removed.

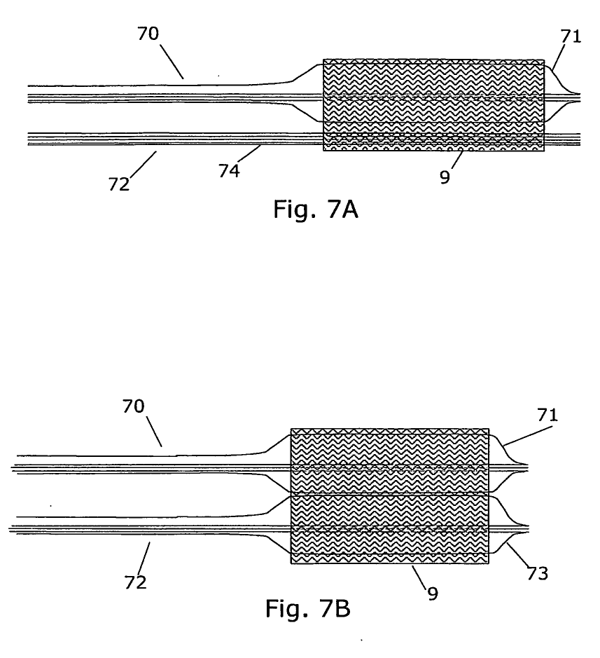

A catheter system providing guidewire lumens for two guidewires through a single stent is represented in FIG. 7A in which two catheters 70 and 72 are placed alongside one another in substantially parallel relationship, the catheter 70 comprising a balloon 71. The individual catheters may be of any known type. An expandable stent 9 is positioned around the balloon 71 of the first catheter 70 while also circumventing part of the lumen 74 of the second catheter 72. The second catheter 72 can of course also comprise a balloon (not shown) which can be positioned distal or proximal to the first catheter's balloon 71.

In FIG. 7B two catheters 70 and 72 are placed alongside one another in substantially parallel relationship with balloons 71 and 73 positioned substantially side-by-side to each other. An expandable stent 9 is positioned around the balloons of the two catheters. Such a configuration can be useful since standard sizes and shapes of catheters, balloons and/or stents can be used even though the diameter of the artery or vessel to be stented is larger than usual or if has an unusual shape. It is therefore not necessary to keep a large stock of differently sized products.

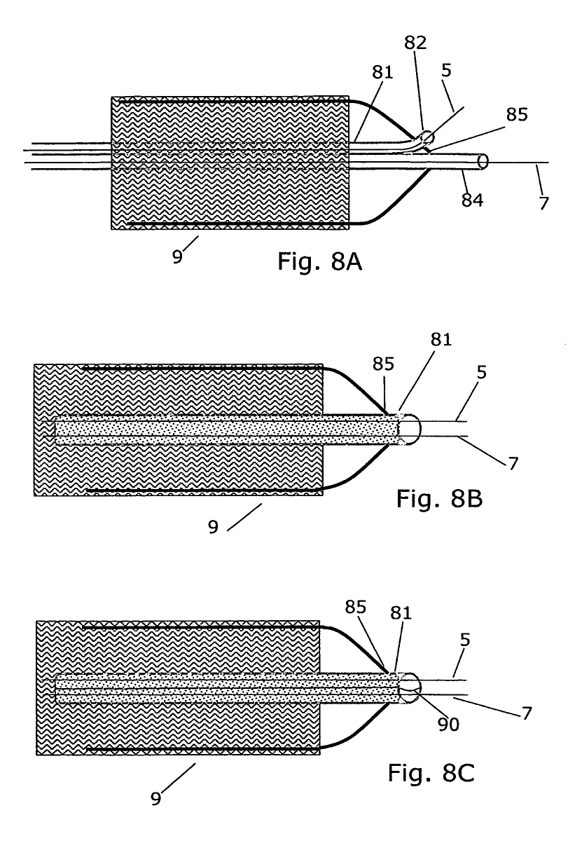

A catheter with a short balloon tip allowing the stent in the principal vessel to be mounted much closer to the bifurcation is illustrated in FIGS. 8A-C. The tip has a high profile, giving the catheter a blunt distal end, which is designated 85 in FIGS. 8A-C. Such a catheter gives the cardiologist the opportunity of keeping guidewires in all branches, while not entering any or only one of the branches with the catheter.

In FIG. 8A the lumen 81 is split into two individual lumens 82 and 84 each defining a lumen for guidewires 5 and 7 respectively. The splitting of the two takes place at a position Inside the catheter. The lumen 82 leaves or exits the catheter at a position proximal to the balloon tip 85. Thereby, the tip of the catheter may enter a branch of a bifurcation up to the exiting point of the other guidewire.

In an alternative embodiment illustrated in FIG. 8B, the lumen 81 is not divided in two and guidewires 5 and 7 are threaded simultaneously in the same lumen.

In yet another embodiment illustrated in FIG. 8C, the guidewires 5 and 7 also share the same lumen but are kept apart by an internal partition 90, extending along the length of the lumen 81.

When the guidewires are threaded in individual lumens as for the catheter in FIG. 8A, the threading may be disturbed if the wires are entangled or twisted in the arteries. In case of the single lumen embodiment also mentioned in the above, this complication during threading does not arise.

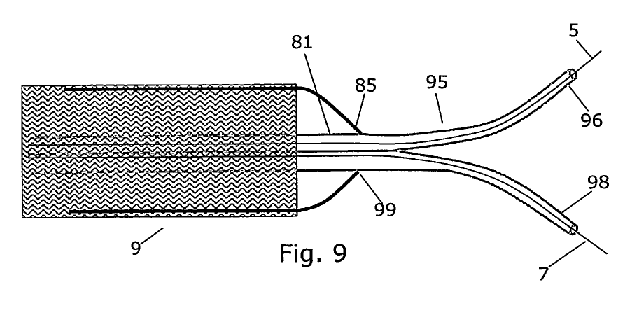

In a further embodiment illustrated in FIG. 9, the catheter has a split tip 95 wherein the lumen(s) 81 emerging from the balloon is/are divided into two or more individual guidewire lumens 96 and 98. The individual lumens are prolonged in the direction distal to the balloon to allow the individual lumens to enter respective branches of the bifurcation. The division preferably happens exactly at the exit 99 of the lumen(s) from the distal end of the balloon or just inside or outside thereof. FIG. 9 shows a situation where the lumens are divided just outside the balloon tip.

The method for stenting bifurcations according to the invention has been described in relation to a simple bifurcation in the above. The underlying principle of threading and advancing a catheter over several guidewires, positioning a first stent with subsequent stents overlapping inside the first stent may be applied in other situations as well. Of particular interest is use of the method for stenting of the left main coronary artery, which with present techniques is considered of unacceptable high risk, due to the high risk of occlusion of one of a the large artery branches of the left main coronary artery. Also other complex anatomies such as trifurcations and double bifurcations can be treated with the invention subsequently using two or several systems, eventually of different sizes.

While the invention herein has been illustrated and described in terms of catheters and method for stenting bifurcated vessels, it will be apparent to those skilled in the art that the catheters herein can be used in the coronary arteries, veins and other arteries throughout the patient's vascular system. Certain dimensions and materials of manufacture have been described herein, and can be modified without departing from the spirit and scope of the invention.

1. A balloon catheter (20, 21) for positioning of a stent (9) in coronary or peripheral angioplasty, the catheter comprising a hollow conduit (22) with an open proximal end (23) and a closed distal end (25) forming exactly one expandable section (24) with an outer surface part adapted to hold a stent and having no bifurcations or side openings, one or more guidewire lumens or grooves (30,34,37) to provide passage for two or more guidewires (5,7) inside an outer perimeter of the expandable section.

2-35. (canceled)

36. A balloon catheter for positioning of a stent in coronary or peripheral angioplasty, the catheter comprising one hollow conduit with an open proximal end and a closed distal end forming exactly one expandable section with an outer surface part adapted to hold a stent and having no bifurcations or side openings, one or more guidewire lumens or grooves to provide passage for two or more guidewires inside an outer perimeter of the expandable section.

37. The balloon catheter according to claim 36, wherein the one or more guidewire lumen(s) provide(s) passage for two or more guidewires inside said outer surface part of the hollow conduit from one or more open end part(s) of the one or more guidewire lumen(s) proximal to said outer surface part and through the closed end of the hollow conduit distal to said outer surface part.

38. The balloon catheter according to claim 36 wherein the catheter is an over-the-wire or a rapid exchange type catheter.

39. A balloon catheter for positioning of a stent in coronary or peripheral angioplasty, the catheter comprising a hollow conduit with an open proximal end and a closed distal end forming an expandable section for holding and expanding a stent,
the balloon catheter being characterised in that
it comprises exactly one expandable section,
it further comprises one or more guidewire lumens or grooves extending along at least part of the expandable section and providing passage for at least two guidewires inside the expandable section so that, after expansion of a stent by the expandable section, the at least two guidewires run through the stent from end to end, and in that
the expandable section has an outer perimeter with no bifurcations or side openings.

40. A balloon catheter for positioning of a stent in coronary or peripheral angioplasty, the catheter comprising a hollow conduit with an open proximal end and a closed distal end forming an expandable section for holding and expanding a stent,
the balloon catheter being characterised in that
it comprises exactly one expandable section,
it further comprises one or more guidewire lumens or grooves extending along at least part of the expandable section and providing passage for at least two guidewires inside the expandable section so that, after expansion of a stent by the expandable section, the at least two guidewires pass through the stent from end to end, and in that
it is adapted to position the stent in a principal vessel proximal to the bifurcation without entering either branch distal to the bifurcation with the expandable section.

41. The balloon catheter according to claim 36, wherein the expandable section comprises a cylindrical central section for holding a stent, and where a distance from the distal end of the cylindrical central section to an inlet of a first guidewire lumen or groove is less than 8 mm.

42. The balloon catheter according to claim 41, wherein said distance is less than 6 mm.

43. The balloon catheter according to claim 41, wherein said distance is less than 2 mm.

44. The balloon catheter according to claim 36, wherein said one or more guidewire lumen(s) extend(s) beyond an extreme distal end of the expandable section and is divided into two or more individual guidewire lumens at a position of exit from the extreme distal end of the expandable section.

45. An assembled stent delivery system comprising a balloon catheter according to claim 36, and a stent held by the expandable section of the hollow conduit so that the one or more guidewire lumen(s) or groove(s) provide(s) inlets and outlets for two or more guidewires distally and proximally to the stent.

46. An assembled stent delivery system comprising two or more balloon catheters extending in parallel to each other and a stent held by and circumventing an expandable section of a first balloon catheter and a non-expandable section of a second balloon catheter, the system thereby providing passage for two or more guidewires through the stent.

47. The assembled stent delivery system according to claim 46, wherein the catheters are over-the-wire and/or rapid exchange type catheters.

48. The assembled stent delivery system according to claim 46, wherein the stent is coated with one or more anti proliferative medical agents.

49. The assembled stent delivery system according to claim 46, wherein the stent is bio degradable.

50. A method for positioning a stent in a principal vessel proximally to a bifurcation, the method comprising the steps of:
inserting a distal end of a first guidewire through the principal vessel and into a first branch of the bifurcation,
inserting a distal end of a second guidewire through the principal vessel and into a second branch of the bifurcation,
providing a first catheter for positioning of a first expandable stent mounted on a distal end section of the catheter, the first catheter comprising one or more guidewire lumen(s) providing passage for two or more wires through the stent from end to end,
threading the one or more guidewire lumen(s) with proximal ends of the first and the second wire,
advancing the first catheter simultaneously over the first and the second wire until the first stent reaches the principal vessel proximal to the bifurcation, and
expanding the first stent.

51. A method for positioning stents at a bifurcation of an artery and in a principal vessel proximally to the bifurcation, the method comprising positioning a stent in the principal vessel proximally to the bifurcation according to claim 50, the method further comprising the steps of:
withdrawing the first catheter simultaneously over the first and the second wire,
threading and advancing a second catheter mounted with a second expandable stent over the first guidewire and at least partially into the first branch of the bifurcation, and
expanding the second stent of the second catheter.

52. The method according to claim 51, wherein the step of advancing the second catheter comprises advancing the second catheter so that a distal end of the second stent is positioned in the first branch of the bifurcation and a proximal end is positioned inside the first stent.

53. The method according to claim 51, further comprising the steps of:
threading and advancing a third catheter mounted with a third expandable stent over the second guidewire and at least partially into the second branch of the bifurcation, and
expanding the third stent of the third catheter.

54. The method according to claim 53, wherein the step of advancing the third catheter comprises advancing the third catheter so that a distal end of the third stent is positioned in the second branch of the bifurcation and a proximal end is positioned inside the first stent.

55. The use a catheter according to claim 36 for performing angioplasty.

56. The catheter according to claim 36 for use in coronary angioplasty on humans.

57. The balloon catheter according to claim 39, wherein the expandable section comprises a cylindrical central section for holding a stent, and where a distance from the distal end of the cylindrical central section to an inlet of a first guidewire lumen or groove is less than 8 mm.

58. The balloon catheter according to claim 57, wherein said distance is less than 6 mm.

59. The balloon catheter according to claim 57, wherein said distance is less than 2 mm.

60. The balloon catheter according to claim 39, wherein said one or more guidewire lumen(s) extend(s) beyond an extreme distal end of the expandable section and is divided into two or more individual guidewire lumens at a position of exit from the extreme distal end of the expandable section.

61. An assembled stent delivery system comprising a balloon catheter according to claim 39 and a stent held by the expandable section of the hollow conduit so that the one or more guidewire lumen(s) or groove(s) provide(s) inlets and outlets for two or more guidewires distally and proximally to the stent.

62. The use a catheter according to claim 26 for performing angioplasty.

63. The catheter according to claim 39 for use in coronary angioplasty on humans.

64. The balloon catheter according to claim 40, wherein the expandable section comprises a cylindrical central section for holding a stent, and where a distance from the distal end of the cylindrical central section to an inlet of a first guidewire lumen or groove is less than 8 mm.

65. The balloon catheter according to claim 64, wherein said distance is less than 6 mm.

66. The balloon catheter according to claim 64, wherein said distance is less than 2 mm.

67. The balloon catheter according to claim 40, wherein said one or more guidewire lumen(s) extend(s) beyond an extreme distal end of the expandable section and is divided into two or more individual guidewire lumens at a position of exit from the extreme distal end of the expandable section.

68. An assembled stent delivery system comprising a balloon catheter according to claim 40 and a stent held by the expandable section of the hollow conduit so that the one or more guidewire lumen(s) or groove(s) provide(s) inlets and outlets for two or more guidewires distally and proximally to the stent.

69. The use a catheter according to claim 40 for performing angioplasty.

70. The catheter according to claim 40 for use in coronary angioplasty on humans.
